(19) United States
(12) Patent Application Publication
(10) Pub. No.: US 2008/0050459 A1
(43) Pub. Date: Feb. 28, 2008

(54) Cosmetic lightening preparation

(75) Inventors: Michelle Elie, New York, NY (US); John Kressaty, Brentwood, TN (US)

Correspondence Address: COHEN PONTANI LIEBERMAN & PAVANE LLP, Suite 1210, 551 Fifth Avenue, New York, NY, 10176, US

(73) Assignee: 3LAB, Inc.

(21) Appl. No.: 11/894,819

(22) Filed: Aug. 22, 2007

Related U.S. Application Data
(60) Provisional application No. 60/839,205, filed on Aug. 22, 2006.

(51) Int. Cl. A61K 36/484; A61K 36/00; A61Q 19/02

(52) U.S. Cl. 424/757; 424/769

(57) ABSTRACT

A cosmetic preparation comprises an effective amount of Phyllanthus Embilca fruit extract and at least one oligopeptide such as Oligopeptide-4 and Oligopepticje-5 (pro-Elastin oligopeptide) The cosmetic preparation may further comprise Licorice extract, and Alpha-arbutin. It has surprisingly been found that this cosmetic preparation displays a beneficial synergistic effect for the combined treatment of fine line and wrinkles and/or skin brightening.

### RELATED APPLICATIONS

This application claims priority from U.S. Provisional Patent Application Ser. No. 60/839,205 which was filed on Aug. 22, 2006.

### BACKGROUND OF THE INVENTION

1. Field of the Invention

The present invention relates to cosmetic preparation such as cream, in particular skin brightening products.

2. Description of the Related Art

The development and use of both anti-wrinkle products and skin brightening products is a common occurrence in the antiaging skin care market. Moreover, the use of some active ingredients intended for the purpose have been in the public domain for some time. Oligopeptides of various kinds are, nowadays, a staple treatment ingredient due to their ability to inhibit the activity of MMP's (matrix metalloproteinases). Examples of MMP's are Collagenase and Elastase who cause the breakdown of both Collagen and Elastin fibers giving rise to the appearance of wrinkles and loss of elasticity. Natural extracts of different sources such as Licorice Extract, Daisy Flower Extract and ingredients like Alpha-Arbutin have been used either singly or in combination to achieve a reduction in the intensity of skin pigmentation achieving evening of the skin tone via various mechanisms.

Nevertheless, there is still a need for a cosmetic preparation of high efficacy for skin whitening and/or anti-wrinkle.

### SUMMARY OF THE INVENTION

Therefore, in accordance with one embodiment of the present invention, we provide a cosmetic preparation comprising an effective amount of Phyllanthus Emblica fruit extract and at least one oligopeptide. Oligopeptides suitable in the present invention should be in a suitable molecular weight so that they are able to act as carriers of Phyllanthus Emblica fruit extract and penetrate skin to maximize the efficacy. For example, the oligopeptides include Oligopeptide-4 (pro-collagen oligopeptide), and Oligopeptide-5 (pro-Elastin oligopeptide). It has surprisingly been found that the use of Phyllanthus Emblica fruit extract—a multi-functional ingredient with non pro-oxidant free radical scavenging, anti-MMP activity and sun screening properties, in combination with an oligopeptide displays a beneficial synergistic effect for the combined treatment of fine line and wrinkles and/or skin brightening.

In accordance with another embodiment of the present invention, we provide a cosmetic preparation comprising an effective amount of Phyllanthus Emblica fruit extract, Licorice extract, and Alpha-arbutin. This combination leads to a surprisingly synergistic efficacy in skin whitening.

Preferably, the cosmetic composition in accordance with the present invention may further contain a combination of an extremely mild emulsifying agent, such as C12-20 Acid PEG 8 Ester, and Coco Glucoside based on an optimized liquid crystalline phase. This combination provides an optimize liquid crystalline structure; it does not produce an exorbitant increase of the viscosity of the final product yet they stabilize the final product very effectively. An extremely mild emulsifying agent is one that does not produce skin irritation; i.e., highly skin compatible and does not affect the enzymatic systems of the skin; i.e., does not affect the skin's metabolism.

The at least one oligopeptide in the present invention may be in an amount of about 0.001% to about 1%, preferably about 0.4% based on the total weight of the cosmetic composition. The Phyllanthus Emblica fruit extract in the present invention may be in an amount of about 0.1% to about 3%, preferably 0.7% to about 3% based on the total weight of the cosmetic composition. The Licorice extract in the present invention may be in an amount of 0.1% to about 5%, preferably 1% to 5% based on the total weight of the cosmetic composition. The Alpha-arbutin in the present invention may be in an amount of 0.5% to 3%, preferably about 0.5% based on the total weight of the cosmetic composition. The extremely mild emulsifying agent in the present invention may be in an amount of about 1% to about 5%, preferably about 2% to about 5% based on the total weight of the cosmetic composition. The Coco Glucoside in the present invention may be in an amount of about 0.01% to about 1% based on the total weight of the cosmetic composition.

In addition to the above ingredients, the cosmetic composition may contain other cosmetically acceptable ingredients, such as emulsion stabilizer/thickening agent, emulsifier, emollient, solvent, and skin softener, and fragrance.

The cosmetic composition in accordance with the present invention may be formulated any suitable form such as cream, lotion, and gel.

The composition of the present invention may be made following standard methods of preparation of oil in water emulsions in the laboratory. That is, the oil phase is prepared separately from the water phase and they are mixed at a specified temperature. Cooled and the labile ingredients are then added.

The final cosmetic composition in accordance with the present invention may have a pH range of about 4 to about 7, preferably about 4 to about 6, more preferably, from about 4.5 to about 5.3.

The various features of novelty which characterize the invention are pointed out with particularity in the claims annexed to and forming a part of the disclosure. For a better understanding of the invention, its operating advantages, and specific objects attained by its use, reference should be had to the described preferred embodiments of the invention.

### DETAILED DESCRIPTION OF THE PRESENTLY PREFERRED EMBODIMENTS

The following examples further illustrate the present invention without limiting it.

The following cosmetic composition is manufactured by a general procedure described above.

| Ingredient | | | |
|---|---|---|---|
| (TRADENAME) | INCI | % Weight | Listed Supplier |
| DEIONIZED WATER | Water | 47.500 | EWL |
| DISODIUM EDTA | Disodium EDTA | 0.050 | UPI/Ruger/Dow/Ashland/ |
| BUTYLENE GLYCOL | Butylene Glycol | 2.000 | Ashland/UPI/Jeen |
| GLYCERIN | Glycerin | 7.200 | Ashland/UPI/Jeen |
| PROPYL GALLATE | Propyl Gallate | 0.100 | UPI |
| ARLASOLV DMI | Dimethyl Isosorbide | 4.000 | Uniquema/Univar/Ruger/UPI |
| PEMULAN TR-2 | Acrylates/C10-30 | 0.100 | Protameen |
| | Alkylacrylate Crosspolymer | | |
| MICROMA 100 | Polymethylmethacrylate | 0.500 | Ikeda |
| NESATOL | C10-18 Triglycerides | 2.500 | Vevy/United Wholesale |
| PELEMOL PTO | Pentaerythrityl | 5.400 | Phoenix |
| | Tetraoctanoate | | |
| KERATOPLAST | Isodecyl Salicylate | 2.500 | Vevy/United Wholesale |
| SHEA BUTTER | Butyrospermum Parkii (Shea | 4.400 | RITA |
| | Butter) | | |
| DC 200/100CST | Dimethicone | 1.000 | Dow |
| | | | Corning/UPI/Ruger/Ashland/Chemcent |
| PELEMOL 899 | Isononyl Isononanoate (and) | 3.000 | Phoenix |
| | Ethylhexyl Isononanoate | | |
| XALIFIN 15 | C12-20 Acid PEG-8 Ester | 4.000 | Vevy/United Wholesale |
| PROTACHEM CS-50 | Cetearyl Alcohol | 3.500 | Protameen |
| MONTANOV 82 | Cetearyl Alcohol (and) Coco | 1.000 | Seppic |
| | Glucoside | | |
| VITAMIN E | Tocopheryl Acetate | 0.100 | RITA |
| ACETATE | | | |
| SIMULGEL NS | Hydroxyethyl | 6.000 | Seppic |
| | Acrylate/Sodium | | |
| | Acryloyldimethyl Taurate | | |
| | Copolymer (and) Squalane | | |
| | (and) Polysorbate 60 | | |
| LUMISKIN | Diacetyl Boldine | 0.200 | Sederma |
| RITA HA 1-C | Sodium Hyaluronate | 0.500 | RITA |
| EMBLICA | Phyllanthus Emblica Fruit | 1.000 | EMD/Ruger |
| | Extract | | |
| BELIDES | Bellis Perennis (Daisy) | 0.100 | CLR/Actives Int'l |
| | Flower Extract | | |
| COLLAGENON | Oligopeptide-4 | 0.200 | Vevy/United Wholesale |
| DERMONECTIN | Oligopeptide-5 | 0.200 | Vevy/United Wholesale |
| GREEN TEA | Camellia Sinensis (Green | 0.100 | Active Organics |
| EXTRACT | tea) Leaf Extract (and) | | |
| | Butylene Glycol (and) Water | | |
| GINKO BILFOBA | Ginko Biloba Leaf Extract | 0.100 | Active Organics |
| EXTRACT BG | (and) Butylene Glycol (and) | | |
| | Water | | |
| LICORICE EXT | Glycyrrhiza Glabra (Licorice) | 0.100 | Active Organics |
| | Root Extract (and) Butylene | | |
| | Glycol (and) Water | | |
| ARBUTIN | Arbutin | 0.500 | Carribean Aloe/Actives Int'l/Ashland |
| DERMOSOFT 1388 | Water (and) Glycerin (and) | 2.000 | Kinetek |
| | Sodium Levulinate (and) | | |
| | Sodium Anisate | | |
| FRAGRANCE | Fragrance | 0.150 | Givaudan |
| UJ003775/00/PURE | | | |
| WHITE | | | |

Efficacy study of a cream composition in accordance with the present invention is shown below.

### Multifaceted Photoaging Improvement Study

1.0 Objective:

To evaluate the efficacy of topical treatments applied on the face and eye area for a period of eight weeks to improve photoaging on the skin. Efficacy was measured with Spectrophotometer, Cutometer, Corneometer, eye area replicas, photography and a Self-assessment Questionnaire.

2.0 Test Material Handling:

On May 29, 2006, four test samples labeled as listed below were received from Englewood Lab and assigned Dermatest Lab Nos. as follows:

| Dermatest Lab No. | Sample Description |
|---|---|
| M06-1 | WW Cream |
| M06-3 | 3Lab Sunblock |

2.1 Handling:

Upon arrival at Dermatest Pty Ltd. each test material is assigned a unique laboratory code number and entered into a daily log identifying the lot number, sample description, sponsor, date received and tests requested.

Samples are retained for a period of three months beyond submission of final report unless otherwise specified by the sponsor or if sample is known to be in support of governmental applications, in which case retained samples are kept two years beyond final report submission.

Sample disposition is conducted in compliance with appropriate federal, state and local ordinances.

3.0 Population Demographics:

Number of subjects enrolled . . . 18

Number of subjects completing study . . . 18

Age Range . . . 39-64

Sex . . . Female . . . 18

Race . . . Caucasian . . . 18
Hispanic . . . 0
Asian . . . 0
   3.1 Standards for Inclusion in a Study:
1. Individuals diagnosed by the investigator as having moderate photoaging with uneven pigmentation.
2. Females between the ages of 35 and 65
3. Individuals willing to discontinue all photoaging and related skin care products.
4. Individuals who have completed a preliminary medical history mandated by Dermatest Pty Ltd.
5. Individuals, who have read, understood and signed an informed consent document relating to the specific type of study to which they are subscribing. Consent forms are kept on file and are available for examination on the premises of Dermatest Pty Ltd. only.
6. Individuals who understand the instructions for use and are willing to cooperate with the program as stated.
7. Individuals free of any dermatological or systemic disorder, or any appearance issue that may interfere with the accurate evaluation and/or results during the course of the study, at the discretion of the Investigator.
8. Individuals free of any acute or chronic disease that might interfere with or increase the risk of study participation.
9. Individuals able to cooperate with the Investigator and the research staff, are willing to have test materials applied according to protocol, and complete the full course of the study.
   3.2 Standards for Exclusion from a Study:
1. Individuals who are under doctor's care.
2. Individuals who are currently taking any medication that may mask or interfere with the test results.
3. Subjects with a history of any form of skin cancer, melanoma, lupus, psoriasis, connective tissue disease, diabetes, chronic skin allergies or any disease that would increase the risk associated with study participation.
4. Individuals who have dermatological disorder or personal appearance issue, which in the opinion of the Investigator would interfere with the accurate evaluation of the individual's face.
5. Individuals with known hypersensitivity to cosmetic products or with any history of sensitivity to cosmetics in general.
6. Individuals who are currently taking medication (topical or oral) which in the opinion of the Investigator would mask or interfere with the results.
7. Individuals who have had any surgical treatment performed on the facial area which will interfere with the test.
8. Individuals who are unwilling or unable to comply with the listed requirements especially discontinuation of all prescription or OTC cosmetic preparations to the face.
9. Individuals who have participated in another clinical trial or experimental drug within the past 30 days.
10. Female volunteers who indicate that they are pregnant, nursing, or planning a pregnancy.
   4.0 Informed Consent:

A signed informed consent, as required by CFR Title 21, Part 50, was obtained from each panelist prior to initiating the study describing reasons for the study, possible adverse effects, associated risks and potential benefits of the treatment and their limits and liability. Each subject was assigned a permanent identification number and completed an extensive medical history form. These forms along with the signed consent forms are available for inspection on the premises of Dermatest Pty Ltd. only.

5.0 Institutional Ethics Committee (IEC):

The Independent Ethics Committee of Dermatest Pty Ltd. consists of 5 or more individuals chosen from within the company for technical expertise and from the local community for Dermatest Pty Ltd. lay interaction. The list of IEC members are kept on file at Dermatest and are available for inspection during the hours of operation.

6.0 Methodology:

Konica Minolta Spectrophotometer CM-2600d

This instrument utilizes the D/8 geometry conforming to CIE No. 15, ISO 7724/1, ASTM E1164, DIN 5033 Tei17, and JIS Z8722-1982 (diffused illumination/8° viewing system) standards, and offers simultaneous SCI (Specular Component Included) and SCE (Specular Component Excluded) measurements. Light from Xenon lamps diffuses on the inner surface of the integrating sphere and illuminates the specimen uniformly. The light reflected by the specimen surface at an angle of 8 degrees to the normal of the surface is received by the specimen-measuring optical system. The diffused light in the integrating sphere is received by the illumination-monitoring optical system and guided to the sensor. The light reflected by the specimen surface and the diffused light are divided into each wavelength component by the specimen-measuring optical system and illumination-monitoring optical sensor, respectively, and then signal proportional to the light intensity of each component is output to the analog processing circuit. By using the outputs from the specimen-measuring optical system and the illumination-monitoring sensor for calculation, compensation for slight fluctuation in spectral characteristics and the intensity of the illumination light is performed. (Double-beam system.)

Courage+Khazaka Cutometer MPA 580

The Cutometer was used to measure the elasticity of the skin surface, using the vacuum principle. This measurement principle is based on suction and elongation. The probe sucks up a defined area of skin surface and records it optically. Analysis of the recorded measurement curves makes it possible to determine the elastic and plastic characteristics of the skin; viscoelasticity. Young skin shows a high degree of elasticity and loses shape only gradually while regaining its original state after the end of the suction procedure. Skin which is healthy, supple and adequately moist will have a higher elasticity than a dry, rough skin. The Cutometer therefore gives a set of measurements which allows us to quantify elastic characteristics.

Courage+Khazaka Corneometer CM 825

The Corneometer is used to determine the moisture content of the skin's surface. It uses a capacitance method based upon the different dielectric constants of water and other materials. The measuring capacitor shows changes of capacitance according to moisture content of samples.

Skin Surface Replica

Skin surface impressions (replicas) were obtained from the crow's feet area of the face at day 0, week 4, and week 8 of product use. The coded skin surface replica specimens were analyzed using Image-Pro Plus software. One can measure changes in skin surface topography by selecting a gray level threshold that allows one to directly determine the projected area of the shadowed region associated with the wrinkles. This parameter, called Shadows, is expressed as a percent of the total area covered by the shadowing. If the surface is rather smooth and flat, there will be few shadows and this value will be small, but if the surface is wrinkled and rough, the shadowed areas will correspondingly increase. In addition, Ra is also computed, which involves generating an average line through the center of the profile and determining the area of deviation above and below this line.

Photo Booth

Photographs were conducted using a photo booth with a 3-point head restraint with photographs taken of the frontal view, 45 degrees to the right, and 45 degrees to the left. The standard chin rest and a three-point adjustable head support ensure proper positioning of the panelist for each time point. Digital Photographs of the face were taken using Nikon Coolpix 8400 Digital Camera at Day 0 (pre-application), four weeks and eight weeks of product usage.

Self-Assessment Questionnaire

Panelists were asked to answer a consumer questionnaire, developed by the sponsor, at four weeks and eight weeks based on their experience with the test product. Questions were based on whether or not an improvement was noticed with fine lines/wrinkles, roughness/dryness, appearance of age spots/freckles/skin discolorations, skin lightening, softness/smoothness, radiance/tone/clarity, firmness/tightness/elasticity, skin moisture, and overall appearance. Answers consisted of strongly agree, somewhat agree, somewhat disagree and strongly disagree.

7.0 Study Design:

Eighteen healthy females between the ages of 39 and 64 were inducted into this study. The panelists applied WW Cream twice a day, morning and night, to cleansed skin as directed for the entire treatment period of the study (8 weeks). They were asked to use 3Lab Sunblock after morning application before going out. At the baseline visit (day 0), all panelists completed the informed consent and medical history forms. Corneometer, Spectrophotometer, Cutometer measurements, photoggraphs and replicas were taken at baseline, week 4, and week 8. The panelists washed their face 30 minutes prior to making the replica to ensure that no make-up, oils or creams were on the skin while obtaining the replica and were asked to remain relaxed and free of any facial expressions in order to prevent alteration in the appearance of the crow's feet area. Panelists were also asked to assess product performance at 4 and 8 weeks of product use by completing a questionnaire designated to evaluate panelist's face for fine lines/wrinkles, roughness/dryness, appearance of age spots/freckles/skin discolorations, skin lightening, softness/smoothness, radiance/tone/clarity, firmness/tightness/elasticity, skin moisture, and overall appearance.

At the completion of the study, all unused study products were collected from the panelists and any adverse events during the study were recorded.

8.0 Results:

Please see attached tables.

9.0 Observations:

No adverse effects or unexpected reactions of any kind were observed on any of the subjects.

10.0 Archiving:

All raw data sheets, technician's notebooks, correspondence files, and copies of final reports are maintained on premises of Dermatest Ply Ltd. in limited access storage files marked “Archive” for five years after completion of the study. A duplicate disk copy of final reports is separately archived in a bank safe deposit vault.

11.0 Conclusions:

Within the limits imposed by the conduct and population size of the study described herein, the test material (Dermatest Lab No.: M06-1, Client No.: WW Cream) demonstrated a significant improvement in the appearance of facial photoaging.

TABLE 1
SPECTROPHOTOMETER READINGS (Day 0, Week 4 & Week 8)
L values (SCI) of Hyper-Pigmented Spot
Lab Nos.: M06-1/M06-3

| | Panelist | | | | | % | | % |
|---|---|---|---|---|---|---|---|---|
| No. | No. | Age | Race | Day 0 | Week 4 | Diff | Week 8 | Diff |
| 1 | M 201 | 46 | C | 52.13 | 52.75 | 1.19 | 52.57 | 0.84 |
| 2 | M 202 | 42 | C | 48.53 | 47.92 | −1.25 | 48.96 | 0.89 |
| 3 | M 203 | 54 | C | 56.80 | 57.80 | 1.77 | 58.33 | 2.70 |
| 4 | M 204 | 43 | C | 54.99 | 58.04 | 5.57 | 58.19 | 5.82 |
| 5 | M 205 | 48 | C | 56.34 | 56.93 | 1.05 | 56.90 | 1.00 |
| 6 | M 206 | 58 | C | 57.69 | 58.01 | 0.55 | 56.95 | −1.28 |
| 7 | M 207 | 40 | C | 55.02 | 55.70 | 1.25 | 56.52 | 2.75 |
| 8 | M 208 | 47 | C | 60.13 | 60.23 | 0.17 | 59.74 | −0.65 |
| 9 | M 209 | 48 | C | 53.75 | 52.71 | −1.92 | 54.99 | 2.32 |
| 10 | M 210 | 49 | C | 47.97 | 47.87 | −0.19 | 48.79 | 1.72 |
| 11 | M 211 | 39 | C | 45.03 | 47.50 | 5.53 | 48.36 | 7.45 |
| 12 | M 212 | 64 | C | 52.18 | 53.24 | 2.04 | 53.70 | 2.93 |
| 13 | M 213 | 58 | C | 52.68 | 53.79 | 2.11 | 54.76 | 3.95 |
| 14 | M 214 | 64 | C | 58.75 | 60.18 | 2.43 | 58.87 | 0.19 |
| 15 | M 215 | 46 | C | 50.56 | 52.76 | 4.35 | 51.82 | 2.49 |
| 16 | M 216 | 48 | C | 52.75 | 54.75 | 3.79 | 52.82 | 0.12 |
| 17 | M 217 | 55 | C | 58.85 | 60.29 | 2.47 | 59.77 | 1.58 |
| 18 | M 218 | 43 | C | 57.72 | 56.04 | −2.91 | 57.64 | −0.14 |

| | | | | | |
|---|---|---|---|---|---|
| MEAN | 53.99 | 54.81 | 1.56 | 54.98 | 1.93 |
| % DIFF | | 1.51 | | 1.83 | |
| t-Stat | | −2.82 | | −3.78 | |
| Statistical Significance | | Signif- | | Signif- | |
| | | icant | | icant | |

*Statistically Significant Critical Value = 1.7396

TABLE 2
SPECTROPHOTOMETER READINGS (Day 0, Week 4 & Week 8)
L values (SCE) of Hyper-Pigmented Spot
Lab Nos.: M06-1/M06-3

| | Panelist | | | | | % | | % |
|---|---|---|---|---|---|---|---|---|
| No. | No. | Age | Race | Day 0 | Week 4 | Diff | Week 8 | Diff |
| 1 | M 201 | 46 | C | 52.07 | 52.55 | 0.93 | 52.38 | 0.60 |
| 2 | M 202 | 42 | C | 48.42 | 47.80 | −1.27 | 48.79 | 0.77 |
| 3 | M 203 | 54 | C | 56.78 | 57.68 | 1.59 | 57.70 | 1.63 |
| 4 | M 204 | 43 | C | 54.79 | 57.96 | 5.80 | 58.07 | 5.99 |
| 5 | M 205 | 48 | C | 56.50 | 56.80 | 0.54 | 56.75 | 0.44 |
| 6 | M 206 | 58 | C | 57.59 | 57.74 | 0.26 | 56.61 | −1.70 |
| 7 | M 207 | 40 | C | 54.91 | 55.71 | 1.46 | 56.51 | 2.92 |
| 8 | M 208 | 47 | C | 60.09 | 60.14 | 0.09 | 59.66 | −0.71 |
| 9 | M 209 | 48 | C | 53.54 | 52.64 | −1.69 | 54.87 | 2.49 |
| 10 | M 210 | 49 | C | 47.81 | 47.71 | −0.19 | 48.64 | 1.75 |
| 11 | M 211 | 39 | C | 45.00 | 47.43 | 5.42 | 48.32 | 7.40 |
| 12 | M 212 | 64 | C | 52.18 | 53.30 | 2.16 | 53.67 | 2.88 |
| 13 | M 213 | 58 | C | 52.37 | 53.60 | 2.35 | 54.44 | 3.96 |
| 14 | M 214 | 64 | C | 58.63 | 60.13 | 2.55 | 58.62 | −0.02 |
| 15 | M 215 | 46 | C | 50.34 | 52.47 | 4.24 | 51.61 | 2.52 |
| 16 | M 216 | 48 | C | 52.67 | 54.77 | 3.98 | 52.70 | 0.05 |
| 17 | M 217 | 55 | C | 58.84 | 60.20 | 2.32 | 59.68 | 1.44 |
| 18 | M 218 | 43 | C | 57.68 | 56.00 | −2.91 | 57.49 | −0.33 |

| | | | | | |
|---|---|---|---|---|---|
| MEAN | 53.90 | 54.70 | 1.54 | 54.81 | 1.78 |
| % DIFF | | 1.49 | | 1.68 | |
| t-Stat | | −2.75 | | −3.29 | |
| Statistical Significance | | Signif- | | Signif- | |
| | | icant | | icant | |

*Statistically Significant Critical Value = 1.7396

TABLE 3
SPECTROPHOTOMETER READINGS (Day 0, Week 4 & Week 8)
L values (SCI) of Surrounding Skin
Lab Nos.: M06-1/M06-3

| | Panelist | | | | | % | | % |
|---|---|---|---|---|---|---|---|---|
| No. | No. | Age | Race | Day 0 | Week 4 | Diff | Week 8 | Diff |
| 1 | M 201 | 46 | C | 58.31 | 60.26 | 3.35 | 60.77 | 4.24 |
| 2 | M 202 | 42 | C | 59.33 | 60.26 | 1.56 | 60.97 | 2.76 |
| 3 | M 203 | 54 | C | 61.87 | 63.09 | 1.98 | 62.14 | 0.43 |
| 4 | M 204 | 43 | C | 66.67 | 67.10 | 0.65 | 65.56 | −1.66 |
| 5 | M 205 | 48 | C | 60.68 | 59.94 | −1.21 | 61.10 | 0.70 |
| 6 | M 206 | 58 | C | 62.32 | 59.94 | −3.81 | 59.94 | −3.82 |
| 7 | M 207 | 40 | C | 65.83 | 64.31 | −2.32 | 63.71 | −3.22 |
| 8 | M 208 | 47 | C | 64.83 | 63.96 | −1.34 | 62.72 | −3.26 |
| 9 | M 209 | 48 | C | 60.80 | 61.48 | 1.13 | 61.50 | 1.16 |
| 10 | M 210 | 49 | C | 55.88 | 56.74 | 1.55 | 57.19 | 2.35 |
| 11 | M 211 | 39 | C | 49.77 | 51.91 | 4.29 | 52.15 | 4.78 |
| 12 | M 212 | 64 | C | 58.58 | 58.69 | 0.19 | 58.39 | −0.32 |
| 13 | M 213 | 58 | C | 59.74 | 59.56 | −0.31 | 59.46 | −0.47 |
| 14 | M 214 | 64 | C | 64.02 | 64.67 | 1.02 | 63.28 | −1.15 |
| 15 | M 215 | 46 | C | 62.67 | 61.38 | −2.06 | 61.64 | −1.64 |
| 16 | M 216 | 48 | C | 59.01 | 60.28 | 2.17 | 60.51 | 2.56 |
| 17 | M 217 | 55 | C | 63.86 | 66.21 | 3.69 | 64.46 | 0.95 |
| 18 | M 218 | 43 | C | 60.07 | 60.41 | 0.58 | 61.07 | 1.66 |

| | | | | | |
|---|---|---|---|---|---|
| MEAN | 60.79 | 61.12 | 0.62 | 60.92 | 0.34 |
| % DIFF | | 0.54 | | 0.21 | |
| t-Stat | | −1.08 | | −0.37 | |
| Statistical Significance | | Signif- | | Signif- | |
| | | icant | | icant | |

*Statistically Significant Critical Value = 1.7396

TABLE 4
SPECTROPHOTOMETER READINGS (Day 0, Week 4 & Week 8)
L values (SCE) of Surrounding Skin
Lab Nos.: M06-1/M06-3

| | Panelist | | | | | % | | % |
|---|---|---|---|---|---|---|---|---|
| No. | No. | Age | Race | Day 0 | Week 4 | Diff | Week 8 | Diff |
| 1 | M 201 | 46 | C | 58.20 | 60.14 | 3.34 | 60.62 | 4.17 |
| 2 | M 202 | 42 | C | 59.29 | 59.96 | 1.14 | 60.81 | 2.58 |
| 3 | M 203 | 54 | C | 61.67 | 62.94 | 2.07 | 61.89 | 0.36 |
| 4 | M 204 | 43 | C | 66.59 | 66.96 | 0.56 | 65.49 | −1.64 |
| 5 | M 205 | 48 | C | 60.56 | 59.87 | −1.13 | 61.00 | 0.73 |
| 6 | M 206 | 58 | C | 62.27 | 59.87 | −3.85 | 59.82 | −3.93 |
| 7 | M 207 | 40 | C | 65.71 | 64.12 | −2.42 | 63.57 | −3.26 |
| 8 | M 208 | 47 | C | 64.81 | 63.93 | −1.35 | 62.71 | −3.24 |
| 9 | M 209 | 48 | C | 60.55 | 61.36 | 1.34 | 61.22 | 1.11 |
| 10 | M 210 | 49 | C | 55.82 | 56.60 | 1.40 | 57.07 | 2.23 |
| 11 | M 211 | 39 | C | 49.58 | 51.70 | 4.27 | 51.96 | 4.80 |
| 12 | M 212 | 64 | C | 58.61 | 58.68 | 0.11 | 58.36 | −0.44 |
| 13 | M 213 | 58 | C | 59.60 | 59.45 | −0.24 | 59.14 | −0.76 |
| 14 | M 214 | 64 | C | 63.88 | 64.48 | 0.95 | 63.03 | −1.33 |
| 15 | M 215 | 46 | C | 62.47 | 61.12 | −2.15 | 61.37 | −1.74 |
| 16 | M 216 | 48 | C | 59.08 | 60.23 | 1.97 | 60.46 | 2.35 |
| 17 | M 217 | 55 | C | 63.87 | 66.14 | 3.55 | 64.45 | 0.91 |
| 18 | M 218 | 43 | C | 59.84 | 60.27 | 0.74 | 60.80 | 1.62 |

| | | | | | |
|---|---|---|---|---|---|
| MEAN | 60.69 | 60.99 | 0.57 | 60.76 | 0.25 |
| % DIFF | | 0.50 | | 0.13 | |
| t-Stat | | −0.99 | | −0.22 | |
| Statistical Significance | | Signif- | | Signif- | |
| | | icant | | icant | |

*Statistically Significant Critical Value = 1.7396

TABLE 5
CORNEOMETER READINGS (Day 0, Week 4 & Week 8)
Lab Nos.: M06-1/M06-3

| | Panelist | | | | Week | % | Week | % |
|---|---|---|---|---|---|---|---|---|
| No. | No. | Age | Race | Day 0 | 4 | Diff | 8 | Diff |
| 1 | M 201 | 46 | C | 52.30 | 56.97 | 8.93 | 59.13 | 13.06 |
| 2 | M 202 | 42 | C | 42.63 | 60.70 | 42.39 | 49.17 | 15.34 |
| 3 | M 203 | 54 | C | 40.13 | 47.07 | 17.29 | 53.37 | 32.99 |
| 4 | M 204 | 43 | C | 50.83 | 51.70 | 1.71 | 40.53 | −20.26 |
| 5 | M 205 | 48 | C | 35.67 | 45.67 | 28.03 | 41.50 | 16.34 |
| 6 | M 206 | 58 | C | 52.20 | 61.13 | 17.11 | 66.90 | 28.16 |
| 7 | M 207 | 40 | C | 31.23 | 42.53 | 36.18 | 50.30 | 61.06 |
| 8 | M 208 | 47 | C | 51.17 | 67.73 | 32.36 | 78.00 | 52.43 |
| 9 | M 209 | 48 | C | 38.03 | 60.70 | 59.61 | 53.57 | 40.86 |
| 10 | M 210 | 49 | C | 21.63 | 45.23 | 109.11 | 46.27 | 113.92 |
| 11 | M 211 | 39 | C | 63.03 | 62.30 | −1.16 | 70.80 | 12.33 |
| 12 | M 212 | 64 | C | 35.20 | 49.90 | 41.76 | 50.27 | 42.81 |
| 13 | M 213 | 58 | C | 41.10 | 50.80 | 23.60 | 64.57 | 57.10 |
| 14 | M 214 | 64 | C | 51.70 | 44.20 | −14.51 | 51.63 | −0.14 |
| 15 | M 215 | 46 | C | 54.67 | 65.10 | 19.08 | 61.97 | 13.35 |
| 16 | M 216 | 48 | C | 48.53 | 68.17 | 40.47 | 68.20 | 40.53 |
| 17 | M 217 | 55 | C | 56.00 | 64.60 | 15.36 | 64.20 | 14.64 |
| 18 | M 218 | 43 | C | 63.17 | 64.70 | 2.42 | 72.97 | 15.51 |

| | | | | | |
|---|---|---|---|---|---|
| MEAN | 46.07 | 56.07 | 26.65 | 57.96 | 30.56 |
| % DIFF | | 21.70 | | 25.82 | |
| t-Stat | | −5.07 | | −5.47 | |
| Statistical Significance | | Sig- | | Sig- | |
| | | nif- | | nif- | |
| | | icant | | icant | |

*Statistically Significant Critical Value = 1.7396

TABLE 6
CUTOMETER READINGS (Day 0, Week 4 & Week 8)
R2 - Gross Elasticity
Lab Nos.: M06-1/M06-3

| No. | Panelist No. | Age | Race | Day 0 | Week 4 | % Diff | Week 8 | % Diff |
|---|---|---|---|---|---|---|---|---|
| 1 | M 201 | 46 | C | 0.5321 | 0.5833 | 9.62 | 0.5729 | 7.67 |
| 2 | M 202 | 42 | C | 0.4531 | 0.5294 | 16.84 | 0.6667 | 47.14 |
| 3 | M 203 | 54 | C | 0.5366 | 0.4563 | −14.96 | 0.5733 | 6.84 |
| 4 | M 204 | 43 | C | 0.6767 | 0.5766 | −14.79 | 0.6804 | 0.55 |
| 5 | M 205 | 48 | C | 0.5302 | 0.5563 | 4.92 | 0.6067 | 14.43 |
| 6 | M 206 | 58 | C | 0.4185 | 0.4846 | 15.79 | 0.5610 | 34.05 |
| 7 | M 207 | 40 | C | 0.6017 | 0.6296 | 4.64 | 0.6056 | 0.65 |
| 8 | M 208 | 47 | C | 0.5370 | 0.5596 | 4.21 | 0.6598 | 22.87 |
| 9 | M 209 | 48 | C | 0.3581 | 0.4087 | 14.13 | 0.6273 | 75.17 |
| 10 | M 210 | 49 | C | 0.5782 | 0.5977 | 3.37 | 0.6349 | 9.81 |
| 11 | M 211 | 39 | C | 0.5806 | 0.4773 | −17.79 | 0.6000 | 3.34 |
| 12 | M 212 | 64 | C | 0.4046 | 0.5437 | 34.38 | 0.4479 | 10.70 |
| 13 | M 213 | 58 | C | 0.5054 | 0.4318 | −14.56 | 0.6632 | 31.22 |
| 14 | M 214 | 64 | C | 0.5246 | 0.6463 | 23.20 | 0.6563 | 25.10 |
| 15 | M 215 | 46 | C | 0.5242 | 0.5244 | 0.04 | 0.6863 | 30.92 |
| 16 | M 216 | 48 | C | 0.5843 | 0.6377 | 9.14 | 0.7122 | 21.89 |
| 17 | M 217 | 55 | C | 0.6036 | 0.6358 | 5.33 | 0.5676 | −5.96 |
| 18 | M 218 | 43 | C | 0.6056 | 0.4737 | −21.78 | 0.6410 | 5.85 |

| | | | | | |
|---|---|---|---|---|---|
| MEAN | 0.5308 | 0.5418 | 3.43 | 0.6202 | 19.01 |
| % DIFF | | 2.07 | | 16.83 | |
| t-Stat | | −0.60 | | −4.68 | |
| Statistical Significance | | Significant | | Significant | |

*Statistically Significant Critical Value = 1.7396

TABLE 7
CUTOMETER READINGS (Day 0, Week 4 & Week 8)
R5 - Net Elasticity
Dermatest Lab Nos.: M06-1/M06-3
Client No.: WW Cream/3Lab Sunblock

| No. | Panelist No. | Age | Race | Day 0 | Week 4 | % Diff | Week 8 | % Diff |
|---|---|---|---|---|---|---|---|---|
| 1 | M 201 | 46 | C | 0.4403 | 0.4884 | 10.92 | 0.5082 | 15.42 |
| 2 | M 202 | 42 | C | 0.3492 | 0.4375 | 25.29 | 0.6042 | 73.02 |
| 3 | M 203 | 54 | C | 0.2979 | 0.3333 | 11.88 | 0.5000 | 67.84 |
| 4 | M 204 | 43 | C | 0.3143 | 0.4054 | 28.99 | 0.5156 | 64.05 |
| 5 | M 205 | 48 | C | 0.3846 | 0.3482 | −9.46 | 0.5345 | 38.98 |
| 6 | M 206 | 58 | C | 0.3486 | 0.5000 | 43.43 | 0.5000 | 43.43 |
| 7 | M 207 | 40 | C | 0.5342 | 0.5294 | −0.90 | 0.5476 | 2.51 |
| 8 | M 208 | 47 | C | 0.3270 | 0.4706 | 43.91 | 0.6066 | 85.50 |
| 9 | M 209 | 48 | C | 0.2886 | 0.3333 | 15.49 | 0.6032 | 109.01 |
| 10 | M 210 | 49 | C | 0.4713 | 0.4746 | 0.70 | 0.5789 | 22.83 |
| 11 | M 211 | 39 | C | 0.5258 | 0.4200 | −20.12 | 0.5902 | 12.25 |
| 12 | M 212 | 64 | C | 0.2870 | 0.3796 | 32.26 | 0.3898 | 35.82 |
| 13 | M 213 | 58 | C | 0.5200 | 0.3896 | −25.08 | 0.6364 | 22.38 |
| 14 | M 214 | 64 | C | 0.3765 | 0.5283 | 40.32 | 0.6571 | 74.53 |
| 15 | M 215 | 46 | C | 0.3816 | 0.5106 | 33.81 | 0.7308 | 91.51 |
| 16 | M 216 | 48 | C | 0.3672 | 0.5056 | 37.69 | 0.5227 | 42.35 |
| 17 | M 217 | 55 | C | 0.4433 | 0.3860 | −12.93 | 0.6349 | 43.22 |
| 18 | M 218 | 43 | C | 0.4158 | 0.4364 | 4.95 | 0.6667 | 60.34 |

| | | | | | |
|---|---|---|---|---|---|
| MEAN | 0.3930 | 0.4376 | 14.51 | 0.5737 | 50.28 |
| % DIFF | | 11.36 | | 46.01 | |
| t-Stat | | −2.17 | | −8.17 | |
| Statistical Significance | | Significant | | Significant | |

*Statistically Significant Critical Value = 1.7396

TABLE 8
CUTOMETER READINGS (Day 0, Week 4 & Week 8)
R6 - Viscoelasticity
Lab Nos.: M06-1/M06-3

| No. | Panelist No. | Age | Race | Day 0 | Week 4 | % Diff | Week 8 | % Diff |
|---|---|---|---|---|---|---|---|---|
| 1 | M 201 | 46 | C | 0.6269 | 0.5349 | −14.68 | 0.5738 | −8.47 |
| 2 | M 202 | 42 | C | 0.5238 | 0.5937 | 13.34 | 0.6875 | 31.25 |
| 3 | M 203 | 54 | C | 0.4539 | 0.6349 | 39.88 | 0.7045 | 55.21 |
| 4 | M 204 | 43 | C | 0.3257 | 0.5000 | 53.52 | 0.5156 | 58.31 |
| 5 | M 205 | 48 | C | 0.4327 | 0.3482 | −19.53 | 0.5345 | 23.53 |
| 6 | M 206 | 58 | C | 0.6881 | 0.9118 | 32.51 | 0.7083 | 2.94 |
| 7 | M 207 | 40 | C | 0.6164 | 0.5882 | −4.57 | 0.6905 | 12.02 |
| 8 | M 208 | 47 | C | 0.3585 | 0.6029 | 68.17 | 0.5902 | 64.63 |
| 9 | M 209 | 48 | C | 0.5369 | 0.5333 | −0.67 | 0.7460 | 38.95 |
| 10 | M 210 | 49 | C | 0.6897 | 0.4746 | −31.19 | 0.6579 | −4.61 |
| 11 | M 211 | 39 | C | 0.5979 | 0.7600 | 27.11 | 0.7213 | 20.64 |
| 12 | M 212 | 64 | C | 0.5043 | 0.5036 | −0.14 | 0.6271 | 24.35 |
| 13 | M 213 | 58 | C | 0.8600 | 0.7143 | −16.94 | 0.7273 | −15.43 |
| 14 | M 214 | 64 | C | 0.4353 | 0.5472 | 25.71 | 0.8286 | 90.35 |
| 15 | M 215 | 46 | C | 0.6316 | 0.7447 | 17.91 | 0.9615 | 52.23 |
| 16 | M 216 | 48 | C | 0.3906 | 0.5506 | 40.96 | 0.5795 | 48.36 |
| 17 | M 217 | 55 | C | 0.4433 | 0.4211 | −5.01 | 0.7619 | 71.87 |
| 18 | M 218 | 43 | C | 0.4059 | 0.7273 | 79.18 | 0.7333 | 80.66 |

| | | | | | |
|---|---|---|---|---|---|
| MEAN | 0.5290 | 0.5940 | 16.98 | 0.6861 | 35.93 |
| % DIFF | | 12.29 | | 29.70 | |
| t-Stat | | −1.88 | | −4.61 | |
| Statistical Significance | | Significant | | Significant | |

*Statistically Significant Critical Value = 1.7396

TABLE 9
CUTOMETER READINGS (Day 0, Week 4 & Week 8)
R7 - Elasticity
Lab Nos.: M06-1/M06-3

| No. | Panelist No. | Age | Race | Day 0 | Week 4 | % Diff | Week 8 | % Diff |
|---|---|---|---|---|---|---|---|---|
| 1 | M 201 | 46 | C | 0.2706 | 0.3182 | 17.59 | 0.3229 | 19.33 |
| 2 | M 202 | 42 | C | 0.2292 | 0.2745 | 19.76 | 0.3580 | 56.20 |
| 3 | M 203 | 54 | C | 0.2049 | 0.2039 | −0.49 | 0.2933 | 43.14 |
| 4 | M 204 | 43 | C | 0.2371 | 0.2703 | 14.00 | 0.3402 | 43.48 |
| 5 | M 205 | 48 | C | 0.2685 | 0.2583 | −3.80 | 0.3483 | 29.72 |
| 6 | M 206 | 58 | C | 0.2065 | 0.2615 | 26.63 | 0.2927 | 41.74 |
| 7 | M 207 | 40 | C | 0.3305 | 0.3333 | 0.85 | 0.3239 | −2.00 |
| 8 | M 208 | 47 | C | 0.2407 | 0.2936 | 21.98 | 0.3814 | 58.45 |
| 9 | M 209 | 48 | C | 0.1878 | 0.2174 | 15.76 | 0.3455 | 83.97 |
| 10 | M 210 | 49 | C | 0.2789 | 0.3218 | 15.38 | 0.3492 | 25.21 |
| 11 | M 211 | 39 | C | 0.3290 | 0.2386 | −27.48 | 0.3429 | 4.22 |
| 12 | M 212 | 64 | C | 0.1908 | 0.2524 | 32.29 | 0.2396 | 25.58 |
| 13 | M 213 | 58 | C | 0.2796 | 0.2273 | −18.71 | 0.3684 | 31.76 |
| 14 | M 214 | 64 | C | 0.2623 | 0.3415 | 30.19 | 0.3594 | 37.02 |
| 15 | M 215 | 46 | C | 0.2339 | 0.2927 | 25.14 | 0.3725 | 59.26 |
| 16 | M 216 | 48 | C | 0.2640 | 0.3261 | 23.52 | 0.3309 | 25.34 |
| 17 | M 217 | 55 | C | 0.3071 | 0.2716 | −11.56 | 0.3604 | 17.36 |
| 18 | M 218 | 43 | C | 0.2958 | 0.2526 | −14.60 | 0.3846 | 30.02 |

| | | | | | |
|---|---|---|---|---|---|
| MEAN | 0.2565 | 0.2753 | 9.25 | 0.3397 | 34.99 |
| % DIFF | | 7.33 | | 32.42 | |
| t-Stat | | −1.66 | | −8.26 | |
| Statistical Significance | | Significant | | Significant | |

*Statistically Significant Critical Value = 1.7396

TABLE 10
ANALYSIS OF REPLICAS
RA North-South
Lab Nos.: M06-1/M06-3

| | Panelist | | | | | % | | % |
|---|---|---|---|---|---|---|---|---|
| No. | No. | Age | Race | Day 0 | Week 4 | Diff | Week 8 | Diff |
| 1 | M 201 | 46 | C | 11.7 | 11.0 | −6.0 | 11.7 | 0.0 |
| 2 | M 202 | 42 | C | 18.8 | 13.4 | −28.7 | 16.9 | −10.1 |
| 3 | M 203 | 54 | C | 31.8 | 30.1 | −5.3 | 28.9 | −9.1 |
| 4 | M 204 | 43 | C | 12.6 | 12.7 | 0.8 | 11.0 | −12.7 |
| 5 | M 205 | 48 | C | 11.9 | 10.8 | −9.2 | 12.1 | 1.7 |
| 6 | M 206 | 58 | C | 21.2 | 19.4 | −8.5 | 19.7 | −7.1 |
| 7 | M 207 | 40 | C | 16.2 | 16.8 | 3.7 | 15.2 | −6.2 |
| 8 | M 208 | 47 | C | 17.7 | 17.2 | −2.8 | 20.4 | 15.3 |
| 9 | M 209 | 48 | C | 20.1 | 18.6 | −7.5 | 17.3 | −13.9 |
| 10 | M 210 | 49 | C | 22.3 | 23.5 | 5.4 | 20.1 | −9.9 |
| 11 | M 211 | 39 | C | 18.7 | 19.1 | 2.1 | 20.4 | 9.1 |
| 12 | M 212 | 64 | C | 20.2 | 17.9 | −11.4 | 15.4 | −23.8 |
| 13 | M 213 | 58 | C | 12.4 | 15.3 | 23.4 | 14.3 | 15.3 |
| 14 | M 214 | 64 | C | 18.1 | 13.8 | −23.8 | 10.8 | −40.3 |
| 15 | M 215 | 46 | C | 14.0 | 11.2 | −20.0 | 14.5 | 3.6 |
| 16 | M 216 | 48 | C | 13.3 | 16.3 | 22.6 | 15.4 | 15.8 |
| 17 | M 217 | 55 | C | 20.4 | 15.3 | −25.0 | 18.5 | −9.3 |
| 18 | M 218 | 43 | C | 15.3 | 15.4 | 0.7 | 14.5 | −5.2 |

| | | | | | |
|---|---|---|---|---|---|
| MEAN | 17.59 | 16.54 | −4.98 | 16.51 | −4.83 |
| % Diff | | −5.98 | | −6.19 | |
| t-Stat | | 1.87 | | 1.83 | |
| Statistical Significance | | Signif- | | Signif- | |
| | | icant | | icant | |

*Statistically Significant Critical Value = 1.7396
*M226 Data removed due to poor replica quality.
**M235 Data removed due to inconsistent facial expression.

TABLE 11
ANALYSIS OF REPLICAS
RA East-West
Lab Nos.: M06-1/M06-3

| No. | Panelist No. | Age | Race | Day 0 | Week 4 | % Diff | Week 8 | % Diff |
|---|---|---|---|---|---|---|---|---|
| 1 | M 201 | 46 | C | 10.9 | 9.9 | −9.2 | 11.9 | 9.2 |
| 2 | M 202 | 42 | C | 14.7 | 11.7 | −20.4 | 12.7 | −13.6 |
| 3 | M 203 | 54 | C | 19.1 | 17.8 | −6.8 | 15.3 | −19.9 |
| 4 | M 204 | 43 | C | 12.5 | 15.3 | 22.4 | 13.7 | 9.6 |
| 5 | M 205 | 48 | C | 11.3 | 11.2 | −0.9 | 14.8 | 31.0 |
| 6 | M 206 | 58 | C | 17.5 | 14.7 | −16.0 | 14.2 | −18.9 |
| 7 | M 207 | 40 | C | 13.6 | 14.9 | 9.6 | 14.3 | 5.1 |
| 8 | M 208 | 47 | C | 16.0 | 15.0 | −6.3 | 14.0 | −12.5 |
| 9 | M 209 | 48 | C | 15.4 | 14.0 | −9.1 | 16.5 | 7.1 |
| 10 | M 210 | 49 | C | 12.0 | 13.6 | 13.3 | 13.2 | 10.0 |
| 11 | M 211 | 39 | C | 15.7 | 15.9 | 1.3 | 15.4 | −1.9 |
| 12 | M 212 | 64 | C | 17.0 | 15.4 | −9.4 | 20.1 | 18.2 |
| 13 | M 213 | 58 | C | 13.3 | 12.7 | −4.5 | 14.3 | 7.5 |
| 14 | M 214 | 64 | C | 13.4 | 17.1 | 27.6 | 14.9 | 11.2 |
| 15 | M 215 | 46 | C | 9.7 | 10.0 | 3.1 | 11.3 | 16.5 |
| 16 | M 216 | 48 | C | 15.1 | 14.5 | −4.0 | 14.0 | −7.3 |
| 17 | M 217 | 55 | C | 16.2 | 19.1 | 17.9 | 13.9 | −14.2 |
| 18 | M 218 | 43 | C | 12.1 | 18.4 | 52.1 | 14.3 | 18.2 |

| | | | | | |
|---|---|---|---|---|---|
| MEAN | 14.19 | 14.51 | 3.37 | 14.38 | 3.08 |
| % Diff | | 2.23 | | 1.29 | |
| t-Stat | | −0.56 | | −0.36 | |
| Statistical Significance | | Significant | | Significant | |

*Statistically Significant Critical Value = 1.7396
*M226 Data removed due to poor replica quality.
**M235 Data removed due to inconsistent facial expression.

TABLE 12
ANALYSIS OF REPLICAS
Shadows North-South
Lab Nos.: M06-1/M06-3

| No. | Panelist No. | Age | Race | Day 0 | Week 4 | % Diff | Week 8 | % Diff |
|---|---|---|---|---|---|---|---|---|
| 1 | M 201 | 46 | C | 31.9 | 34.8 | 9.1 | 34.6 | 8.5 |
| 2 | M 202 | 42 | C | 17.2 | 8.8 | −48.8 | 14.9 | −13.4 |
| 3 | M 203 | 54 | C | 34.4 | 32.0 | −7.0 | 29.7 | −13.7 |
| 4 | M 204 | 43 | C | 9.9 | 8.2 | −17.2 | 8.3 | −16.2 |
| 5 | M 205 | 48 | C | 5.9 | 6.4 | 8.5 | 7.6 | 28.8 |
| 6 | M 206 | 58 | C | 21.7 | 18.6 | −14.3 | 22.1 | 1.8 |
| 7 | M 207 | 40 | C | 14.5 | 15.6 | 7.6 | 14.1 | −2.8 |
| 8 | M 208 | 47 | C | 20.0 | 16.0 | −20.0 | 21.6 | 8.0 |
| 9 | M 209 | 48 | C | 23.1 | 20.4 | −11.7 | 19.2 | −16.9 |
| 10 | M 210 | 49 | C | 21.4 | 24.1 | 12.6 | 21.1 | −1.4 |
| 11 | M 211 | 39 | C | 20.9 | 22.1 | 5.7 | 24.5 | 17.2 |
| 12 | M 212 | 64 | C | 22.6 | 18.4 | −18.6 | 16.5 | −27.0 |
| 13 | M 213 | 58 | C | 10.4 | 15.7 | 51.0 | 12.5 | 20.2 |
| 14 | M 214 | 64 | C | 20.0 | 17.4 | −13.0 | 16.4 | −18.0 |
| 15 | M 215 | 46 | C | 13.8 | 9.3 | −32.6 | 16.5 | 19.6 |
| 16 | M 216 | 48 | C | 22.8 | 19.7 | −13.6 | 17.6 | −22.8 |
| 17 | M 217 | 55 | C | 24.9 | 19.0 | −23.7 | 21.9 | −12.0 |
| 18 | M 218 | 43 | C | 17.6 | 20.0 | 13.6 | 17.6 | 0.0 |

| | | | | | |
|---|---|---|---|---|---|
| MEAN | 19.61 | 18.14 | −6.24 | 18.71 | −2.22 |
| % Diff | | −7.50 | | −4.62 | |
| t-Stat | | 1.76 | | 1.27 | |
| Statistical Significance | | Significant | | Significant | |

*Statistically Significant Critical Value = 1.7396
*M226 Data removed due to poor replica quality.
**M235 Data removed due to inconsistent facial expression.

TABLE 13
ANALYSIS OF REPLICAS
Shadows East-West
Lab Nos.: M06-1/M06-3

| No. | Panelist No. | Age | Race | Day 0 | Week 4 | % Diff | Week 8 | % Diff |
|---|---|---|---|---|---|---|---|---|
| 1 | M 201 | 46 | C | 29.7 | 30.2 | 1.7 | 33.4 | 12.5 |
| 2 | M 202 | 42 | C | 11.7 | 6.7 | −42.7 | 8.9 | −23.9 |
| 3 | M 203 | 54 | C | 18.6 | 17.5 | −5.9 | 13.4 | −28.0 |
| 4 | M 204 | 43 | C | 9.1 | 5.4 | −40.7 | 10.2 | 12.1 |
| 5 | M 205 | 48 | C | 6.4 | 8.4 | 31.3 | 11.6 | 81.3 |
| 6 | M 206 | 58 | C | 16.3 | 12.7 | −22.1 | 12.7 | −22.1 |
| 7 | M 207 | 40 | C | 11.7 | 13.6 | 16.2 | 12.3 | 5.1 |
| 8 | M 208 | 47 | C | 15.9 | 13.4 | −15.7 | 12.2 | −23.3 |
| 9 | M 209 | 48 | C | 17.4 | 13.1 | −24.7 | 19.1 | 9.8 |
| 10 | M 210 | 49 | C | 10.7 | 14.5 | 35.5 | 13.4 | 25.2 |
| 11 | M 211 | 39 | C | 17.3 | 18.1 | 4.6 | 17.4 | 0.6 |
| 12 | M 212 | 64 | C | 18.6 | 18.9 | 1.6 | 21.5 | 15.6 |
| 13 | M 213 | 58 | C | 13.3 | 9.4 | −29.3 | 13.6 | 2.3 |
| 14 | M 214 | 64 | C | 13.4 | 25.9 | 93.3 | 23.6 | 76.1 |
| 15 | M 215 | 46 | C | 7.3 | 9.2 | 26.0 | 12.1 | 65.8 |
| 16 | M 216 | 48 | C | 25.5 | 16.4 | −35.7 | 16.3 | −36.1 |
| 17 | M 217 | 55 | C | 22.2 | 24.5 | 10.4 | 19.5 | −12.2 |
| 18 | M 218 | 43 | C | 11.9 | 23.5 | 97.5 | 18.5 | 55.5 |

| | | | | | |
|---|---|---|---|---|---|
| MEAN | 15.39 | 15.63 | 5.62 | 16.09 | 12.01 |
| % Diff | | 1.59 | | 4.58 | |
| t-Stat | | −0.19 | | −0.64 | |
| Statistical Significance | | Significant | | Significant | |

*Statistically Significant Critical Value = 1.7396
*M226 Data removed due to poor replica quality.
**M235 Data removed due to inconsistent facial expression.

TABLE 14
Panelist Questionnaire
4 Week Data
Lab Nos.: M06-1/M06-3

| | Strongly | Somewhat | Somewhat | Strongly | Overall |
|---|---|---|---|---|---|
| Statement | Agree | Agree | Disagree | Disagree | Agreement |
| Significantly reduces the | 1 | 5 | 9 | 3 | 33% |
| appearance of fine lines | | | | | |
| and wrinkles | | | | | |
| Significantly reduces | 1 | 11 | 4 | 2 | 67% |
| roughness and dryness | | | | | |
| Significantly diminishes the | 1 | 8 | 6 | 3 | 50% |
| appearance of age spots, | | | | | |
| freckles, and skin | | | | | |
| discolorations | | | | | |
| Significantly lightens the | 1 | 6 | 10 | 1 | 39% |
| skin | | | | | |
| Significantly improves | 3 | 8 | 6 | 1 | 61% |
| skin's softness and | | | | | |
| smoothness | | | | | |
| Significantly improves | 1 | 6 | 9 | 2 | 39% |
| skin's radiance, tone, and | | | | | |
| clarity | | | | | |
| Significantly improves | 0 | 7 | 11 | 0 | 39% |
| skin's firmness, tightness, | | | | | |
| and elasticity | | | | | |
| Significantly moisturizes | 3 | 11 | 3 | 1 | 78% |
| the skin | | | | | |
| Significantly improves | 1 | 9 | 7 | 1 | 56% |
| skin's overall appearance | | | | | |

Note:
1) “Overall Agreement” is expressed as the total number of panelists answering strongly agree and somewhat agree, divided by number of panelists answering the questionnaire (N = 18), multiplied by 100.
2) Data depicted in Agree/Disagree columns are the number of panelists answering the question.

TABLE 15
Panelist Questionnaire
8 Week Data
Lab Nos.: M06-1/M06-3

| | Strongly | Somewhat | Somewhat | Strongly | Overall |
|---|---|---|---|---|---|
| Statement | Agree | Agree | Disagree | Disagree | Agreement |
| Significantly reduces the | 3 | 10 | 4 | 1 | 72% |
| appearance of fine lines | | | | | |
| and wrinkles | | | | | |
| Significantly reduces | 6 | 7 | 5 | 0 | 72% |
| roughness and dryness | | | | | |
| Significantly diminishes the | 3 | 7 | 7 | 1 | 56% |
| appearance of age spots, | | | | | |
| freckles, and skin | | | | | |
| discolorations | | | | | |
| Significantly lightens the | 4 | 7 | 7 | 0 | 61% |
| skin | | | | | |
| Significantly improves | 7 | 8 | 3 | 0 | 83% |
| skin's softness and | | | | | |
| smoothness | | | | | |
| Significantly improves | 4 | 10 | 4 | 0 | 78% |
| skin's radiance, tone, and | | | | | |
| clarity | | | | | |
| Significantly improves | 0 | 14 | 4 | 0 | 78% |
| skin's firmness, tightness, | | | | | |
| and elasticity | | | | | |
| Significantly moisturizes | 10 | 4 | 4 | 0 | 78% |
| the skin | | | | | |
| Significantly improves | 5 | 10 | 3 | 0 | 83% |
| skin's overall appearance | | | | | |

Note:
1) “Overall Agreement” is expressed as the total number of panelists answering strongly agree and somewhat agree, divided by number of panelists answering the questionnaire (N = 18), multiplied by 100.
2) Data depicted in Agree/Disagree columns are the number of panelists answering the question.

### APPENDIX I

### Spectrophotometer Measurements

This instrument utilizes the D/8 geometry conforming to CIE No. 15, ISO 7724/1, ASTM E1164, DIN 5033 Tei17, and JIS Z8722-1982 (diffused illumination/8° viewing system) standards, and offers simultaneous SCI (specular component included) and SCE (specular component excluded) measurements. Light from xenon lamps diffuses on the inner surface of the integrating sphere and illuminates the specimen uniformly. The light reflected by the specimen surface at an angle of 8 degrees to the normal of the surface is received by the specimen-measuring optical system. The diffused light in the integrating sphere is received by the illumination-monitoring optical system and guided to the sensor. The light reflected by the specimen surface and the diffused light are divided into each wavelength component by the specimen-measuring optical system and illumination-monitoring optical sensor, respectively, and then signal proportional to the light intensity of each component are output to the analog processing circuit. By using the outputs from the specimen-measuring optical system and the illumination-monitoring sensor for calculation, compensation for slight fluctuation in spectral characteristics and the intensity of the illumination light is performed. (Double-beam system)

Any increase in the a* value is indicative of a reddening color and a decrease drives the color toward the green shade. An increase in the b* value indicates yellow enhancement and a decrease signifies a color shift into the blue region as is perceived with a blue coefficient. An increase in the L* value indicates lightening of the color and any diminution of the L* value is indicative of darkening of the color.

| | a*-value | b*-value | L*-value |
|---|---|---|---|
| Increase | Reddening | Yellow | Lightening |
| Decrease | Green | Blue | Darkening |

### APPENDIX II

### Corneometer Measurements

The Corneometer is used to determine the moisture content of the skin's surface. It uses a capacitance method based upon the different dielectric constants of water and other materials. The measuring capacitor shows changes of capacitance according to moisture content of samples.

| | INNER |
|---|---|
| | FOREARM |
| VERY DRY | <30 |
| DRY | 30-45 |
| SUFFICIENTLY | >45 |
| MOISTURIZED | |

This interpretation should be valid for other body parts as well.

### APPENDIX III

### Cutometer Measurements

Explanation of Parameters:

| | |
|---|---|
| R0 | Represents the maximum amplitude on the first curve. The number serves |
| | as an implication of the firmness of the skin. |
| R1 | Represents the minimum amplitude of the first curve. The number indicates |
| | the ability of the skin to return to the original state. |
| R2 | Represents the gross elasticity (ability of redeformation of the skin) of the |
| | skin. The closer to 1 it is the more elastic it is. |
| R3 | Represents the tiring effects on the skin. The comparison of the last |
| | maximum amplitude to the first maximum amplitude, a smaller difference |
| | indicates a smaller tiring effect (amplitude increases with each new suction). |
| R4 | Represents the tiring effects on the skin. The comparison of the last minimum |
| | amplitude to the first minimum amplitude, a smaller difference indicates a |
| | smaller tiring effect (redeformation decreases with each new suction). |
| R5 | Represents the net elasticity. The closer the value is to 1, the greater the |
| | elasticity. |
| R6 | Represents the viscoelasticity. The smaller the value the higher the elasticity. |
| R7 | Represents the elastic portion of the curve compared to the entire curve. The |
| | closer the value is to 1 the more elastic the curve is. |
| R8 | Represents the amplitude of the relaxation time. The closer the values of R8 |
| | and R0 are too each other the greater the ability of the skin to return to its |
| | original state. |
| R9 | Represents the tiring of the skin after repeatedly being suctioned in. The |
| | smaller the value of R9 the smaller the tiring effects. |
| F0 | Represents the maximum amplitude multiplied by the suction time. The closer |
| | the value is to 0 the more elastic the skin is. |
| F1 | Represents the maximum amplitude multiplied by the relaxation time. The |
| | closer the value is to 0 the more elastic the skin is. |
| F4 | Represents the area within and above the envelope curve. The smaller the |
| | value the firmer the skin. F4 utilizes the calculations for F2 and F3. |

The invention is not limited by the embodiments described above which are presented as examples only but can be modified in various ways within the scope of protection defined by the appended patent claims.

We claim:

1- A cosmetic whitening composition comprising an effective amount of Phyllanthus Emblica fruit extract and at least one oligopeptide.

2- The cosmetic whitening composition of claim 1 where in the at least one oligopeptide is selected from the groups consisting of Oligopeptide-4 and Oligopeptide-5.

3- The cosmetic whitening composition of claim 1 further comprising a combination of an extremely mild emulsifying agent and Coco Glucoside based on an optimized liquid crystalline phase.

4- The cosmetic whitening composition of claim 3 wherein the extremely mild emulsifying agent is a C12-20 acid PEG 8 ester.

5- The cosmetic whitening composition of claim 1 wherein the Phyllanthus Emblica fruit extract is in an amount of about 0.1% to about 3% based on the total weight of the cosmetic whitening composition.

6- The cosmetic whitening composition of claim 1 wherein the Phyllanthus Emblica fruit extract is in an amount of 0.7% to about 3% based on the total weight of the cosmetic whitening composition.

7- The cosmetic whitening composition of claim 1 wherein the at least one oligopeptide is in an amount of about 0.001% to about 1% based on the total weight of the cosmetic whitening composition.

8- The cosmetic whitening composition of claim 1 wherein the at least one oligopeptide is in an amount of about 0.4% based on the total weight of the cosmetic whitening composition.

9- The cosmetic whitening composition of claim 3 wherein the extremely mild emulsifying agent is in an amount of about 1% to about 5% based on the total weight of the cosmetic whitening composition.

10- The cosmetic whitening composition of claim 3 wherein the extremely mild emulsifying agent is in an amount of about 2% to about 5% based on the total weight of the cosmetic whitening composition.

11- The cosmetic whitening composition of claim 1 further comprising Licorice extract and Alpha-arbutin.

12- The cosmetic whitening composition of claim 1 wherein the Licorice extract is in an amount of about 0.1% to about 5% based on the total weight of the cosmetic whitening composition.

13- The cosmetic whitening composition of claim 1 wherein the Licorice extract is in an amount of about 0.1% based on the total weight of the cosmetic whitening composition.

14- The cosmetic whitening composition of claim 1 wherein the Alpha-arbutin is in an amount of about 0.1% to about 3% based on the total weight of the cosmetic whitening composition.

15- The cosmetic whitening composition of claim 1 wherein the Alpha-arbutin is in an amount of about 0.5% to about 3% based on the total weight of the cosmetic whitening composition.

16- The cosmetic whitening composition of claim 1 wherein the Alpha-arbutin is in an amount of about 0.5% based on the total weight of the cosmetic whitening composition.

17- The cosmetic whitening composition of claim 1 having a pH range of about 4 to 7.

18- The cosmetic whitening composition of claim 1 having a pH range of about 4 to 6.

19- The cosmetic whitening composition of claim 1 having a pH of 4.5 to 5.3.

20- A cosmetic whitening composition comprising an effective amount of Phyllanthus Emblica fruit extract, Licorice extract, and Alpha-arbutin.

21- The cosmetic whitening composition of claim 20 further comprising at least one oligopeptide.
